(19) United States
(12) Patent Application Publication
(10) Pub. No.: US 2014/0288013 A1
(43) Pub. Date: Sep. 25, 2014

(54) METHODS AND COMPOSITIONS FOR THE MANAGEMENT OF CANCER USING 2-DG AND AN IGF-IR INHIBITOR

(71) Applicant: EMORY UNIVERSITY, Atlanta, GA (US)

(72) Inventors: Wei Zhou, Lilburn, GA (US); Diansheng Zhong, Lilburn, GA (US)

(73) Assignee: EMORY UNIVERSITY, Atlanta, GA (US)

(21) Appl. No.: 14/350,817

(22) PCT Filed: Oct. 17, 2012
(86) PCT No.: PCT/US12/60490

§ 371 Date: Apr. 10, 2014

Related U.S. Application Data
(60) Provisional application No. 61/548,450, filed on Oct. 18, 2011.

(51) Int. Cl. A61K 31/7004; A61K 31/53

(52) U.S. Cl. CPC A61K 31/7004; A61K 31/53
USPC 514/23

(57) ABSTRACT

The disclosure relates to methods and compositions for the management of cancer. In certain embodiments, the disclosure relates to methods of treating or preventing cancer by administering an IGF-IR inhibitor and 2-deoxyglucose to a subject in need thereof.

### CROSS REFERENCE TO RELATED APPLICATIONS

This application claims priority to U.S. Provisional Application No. 61/548,450 filed Oct. 18, 2011, hereby incorporated by reference in its entirety.

### ACKNOWLEDGEMENTS

This invention was made with government support under Grant P01 CA116676 awarded by the National Institute of Health. The government has certain rights in the invention.

### FIELD

The disclosure relates to methods and compositions for the management of cancer. In certain embodiments, the disclosure relates to methods of treating or preventing cancer by administering an IGF-IR inhibitor and 2-deoxyglucose to a subject in need thereof.

### BACKGROUND

Insulin-like growth factor-I receptor (IGF-IR) is a transmembrane heterotetrameric protein. The binding of the ligands, such as insulin-like growth-factor-I (IGF-I) and insulin-like growth factor-II (IGF-II), by the extracellular domain of IGF-IR receptor activates it, and in many instances its intracellular tyrosine kinase domain, resulting in autophosphorylation of the receptor and substrate phosphorylation. IGF-IR is homologous to the insulin receptor, having a sequence similarity of 84% in the beta chain tyrosine kinase domain and a sequence similarity of 48% in the alpha chain extracellular cysteine rich domain (Ulrich, A. et al., 1986, EMBO, 5, 2503-2512; Fujita-Yamaguchi, Y. et al., 1986, J. Biol. Chem., 261, 16727-16731; LeRoith, D. et al., 1995, Endocrine Reviews, 16, 143-163). The IGF-IR and ligands (IGF-I and IGF-II) play important roles in numerous physiological processes including growth and development during embryogenesis, metabolism, cellular proliferation and cell differentiation (LeRoith, D., 2000, Endocrinology, 141, 1287-1288; LeRoith, D., 1997, New England J. Med., 336, 633-640).

IGF-IR has been implicated in promoting growth, transformation and survival of tumor cells (Baserga, R. et al., 1997, Biochem. Biophys. Acta, 1332, F105-F126; Blakesley, V. A. et al., 1997, Journal of Endocrinology, 152, 339-344; Kaleko, M., Rutter, W. J., and Miller, A. D. 1990, Mol. Cell. Biol., 10, 464-473). Several types of tumors are known to express higher than normal levels of IGF-IR, including breast cancer, colon cancer, ovarian carcinoma, synovial sarcoma and pancreatic cancer. See Khandwala, H. M. et al., 2000, Endocrine Reviews, 21, 215-244; Werner, H. and LeRoith, D., 1996, Adv. Cancer Res., 68, 183-223; Happerfield, L. C. et al., 1997, J. Pathol., 183, 412-417; Frier, S. et al., 1999, Gut, 44, 704-708; van Dam, P. A. et al., 1994, J. Clin. Pathol., 47, 914-919; Xie, Y. et al., 1999, Cancer Res., 59, 3588-3591; Bergmann, U. et al., 1995, Cancer Res., 55, 2007-2011.

In vitro, IGF-I and IGF-II have been shown to be potent mitogens for several human tumor cell lines such as lung cancer, breast cancer, colon cancer, osteosarcoma and cervical cancer (Ankrapp, D. P. and Bevan, D. R., 1993, Cancer Res., 53, 3399-3404; Cullen, K. J., 1990, Cancer Res., 50, 48-53; Hermanto, U. et al., 2000, Cell Growth & Differentiation, 11, 655-664; Guo, Y. S. et al., 1995, J. Am. Coll. Surg., 181, 145-154; Kappel, C. C. et al., 1994, Cancer Res., 54, 2803-2807; Steller, M. A. et al., 1996, Cancer Res., 56, 1761-1765). Several of these tumors and tumor cell lines also express high levels of IGF-I or IGF-II, which may stimulate their growth in an autocrine or paracrine manner (Quinn, K. A. et al., 1996, J. Biol. Chem., 271, 11477-11483).

Epidemiological studies have shown a correlation of elevated plasma level of IGF-I (and lower level of IGF-binding protein-3) with increased risk for prostate cancer, colon cancer, lung cancer and breast cancer (Chan, J. M. et al., 1998, Science, 279, 563-566; Wolk, A. et al., 1998, J. Natl. Cancer Inst., 90, 911-915; Ma, J. et al., 1999, J. Natl. Cancer Inst., 91, 620-625; Yu, H. et al., 1999, J. Natl. Cancer Inst., 91, 151-156; Hankinson, S. E. et al., 1998, Lancet, 351, 1393-1396). Strategies to lower the IGF-I level in plasma or to inhibit the function of IGF-IR have been suggested for cancer prevention (Wu, Y. et al., 2002, Cancer Res., 62, 1030-1035; Grimberg, A and Cohen P., 2000, J. Cell. Physiol., 183, 1-9).

While IGF-IR has attractive biology as a therapeutic target, attempts to make antagonists or other types of inhibitors have been slow, largely due to one or more of the following factors depending on the therapeutic paradigm: difficulty or inability to antagonize the target without agonism; difficulty or inability to make selective antagonists or inhibitors due to undesired cross reactivity with other tyrosine kinase targets or receptors, such as the insulin receptor; difficulty in effective administration due to short half life of the potential therapeutic; difficulty in effective administration clue to low solubility of the potential therapeutic; and difficulty in effective administration due to aggregation of the potential therapeutic. See Published US App. No. 2010/0121033 and U.S. Pat. No. 5,840,673. Thus, there is a need to identify preferred therapeutic strategies.

Aerobic glycolysis plays an important role in tumorigenesis and is a valid target for cancer therapy. 2-Deoxyglucose (2-DG) is a glycolytic inhibitor. It also activates a prosurvival oncoprotein, AKT, through IGF1R and PI3K. 2-Deoxyglucose (2-DG) treatments disrupted the binding between insulin-like growth factor I (IGF-I) and IGF-binding protein 3 (IGFBP3). 2-DG-induced activation of many survival pathways can be jointly attenuated through IGF-IR inhibition. Treatment with a combination of 2-DG and the IGF1R inhibitor II reduced cancer cell proliferation and promoted significant apoptosis. See Zhong et al., 2009, J Biol. Chem. 284(35) 23225-23233.

### SUMMARY

The disclosure relates to methods of treating or preventing cancer comprising administering to a subject a therapeutically effective amount of a pharmaceutical compositions comprising 2-deoxyglucose or derivative in combination with an IGF-IR inhibitor such as a 1-(4-aminopyrrolo[2,1-f][1,2,4]triazin-2-yl)pyrrolidine-2-carboxamide derivative.

In certain embodiments, the derivative is a compound of formula I,

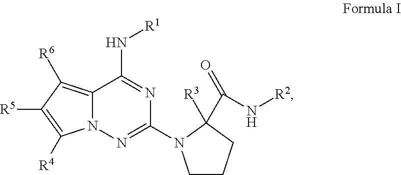

prodrugs, esters, or salts thereof wherein,

R¹, R², R³, R⁴, R⁵, and R⁶ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R¹, R², R³, R⁴, R⁵, and R⁶ are optionally substituted with one or more, the same or different, R⁷;

R⁷ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R⁷ is optionally substituted with one or more, the same or different, R⁸; and

R⁸ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the IGF-IR inhibitor is 1-(4-((5-cyclopropyl-1H-pyrazol-3-yl)amino)pyrrolo[2,1-f][1,2,4]triazin-2-yl)-N-(6-fluoropyridin-3-yl)-2-methylpyrrolidine-2-carboxamide (BMS-754807) optionally substituted with one or more substituents or salts thereof.

In further embodiments, the 2-deoxyglucose and the IGF-IR inhibitor are in the same pharmaceutical composition or administered in separate compositions. In certain embodiments, the subject is at risk of, exhibiting symptom of, or diagnosed with breast cancer, colon cancer, prostate cancer, ovarian carcinoma, synovial sarcoma, pancreatic cancer, lung cancer, osteosarcoma, or cervical cancer.

In certain embodiments, the 2-deoxyglucose derivative is 2-deoxyglucose substituted with one or more, the same or different, substituents.

In certain embodiments, the disclosure relates to a pharmaceutical composition comprising 2-deoxyglucose or derivative and an IGF inhibitor and a pharmaceutically acceptable excipient.

In certain embodiments, the subject is a human subject wherein about 20, 30, 40, 50, 100, 150, 200, 500 mg of 2-deoxyglucose is administered daily and about 20, 30, 40, 50, 100, 150, 200, 500 mg of 1-(4-((5-cyclopropyl-1H-pyrazol-3-yl)amino)pyrrolo[2,1-f][1,2,4]triazin-2-yl)-N-(6-fluoropyridin-3-yl)-2-methylpyrrolidine-2-carboxamide is administered daily.

### BRIEF DESCRIPTION OF THE FIGURES

FIG. 1 shows dose response curves of BMS754807 and 2-DG in LKB1-null, H460, A549, and H157 NSCLC cell lines. 2000 cells were seed in each well of a 96-well plate, and exposed to BMS754807 or 2-DG at indicated concentration for 48 hrs.

FIG. 2 shows data indicating BMS754807 blocked 2-DG induces IGF1R and Akt phosphorylation.

FIG. 3 shows data indicating the combination of BMS754807 and 2-DG induces caspase-3 cleavage.

### DETAILED DESCRIPTION

### IGF-IR Inhibitors

The chemical structure and a method of preparing BMS-754807 is disclosed in Witterman et al, Discovery of a 2,4-Disubstituted Pyrrolo-[1,2-f][1,2,4]triazine Inhibitor (BMS-754807) of Insulin-like Growth Factor Receptor (IGF-1R) Kinase in Clinical Development, J. Med. Chem., 2009, 52 (23), pp 7360-7363 hereby incorporated by reference.

In certain embodiments, the disclosure contemplates the treatment of cancer comprising administering an effective amount of a 2-DG and IGF-1R inhibitor such as a 1-(4-aminopyrrolo[2,1-f][1,2,4]triazin-2-yl)pyrrolidine-2-carboxamide derivative of salt thereof to a subject in need thereof.

In certain embodiments, the derivative is a compound of formula I,

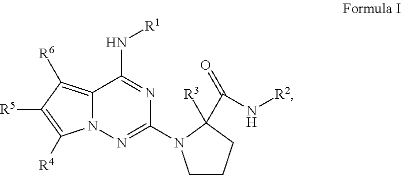

prodrugs, esters, or salts thereof wherein,

R¹, R², R³, R⁴, R⁵, and R⁶ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R¹, R², R³, R⁴, R⁵, and R⁶ are optionally substituted with one or more, the same or different, R⁷;

R⁷ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R⁷ is optionally substituted with one or more, the same or different, R⁸; and

R⁸ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the IGF-IR inhibitor is 1-(4-((5-cyclopropyl-1H-pyrazol-3-yl)amino)pyrrolo[2,1-f][1,2,4]triazin-2-yl)-N-(6-fluoropyridin-3-yl)-2-methylpyrrolidine-2-carboxamide (BMS-754807) optionally substituted with one or more substituents or salts thereof.

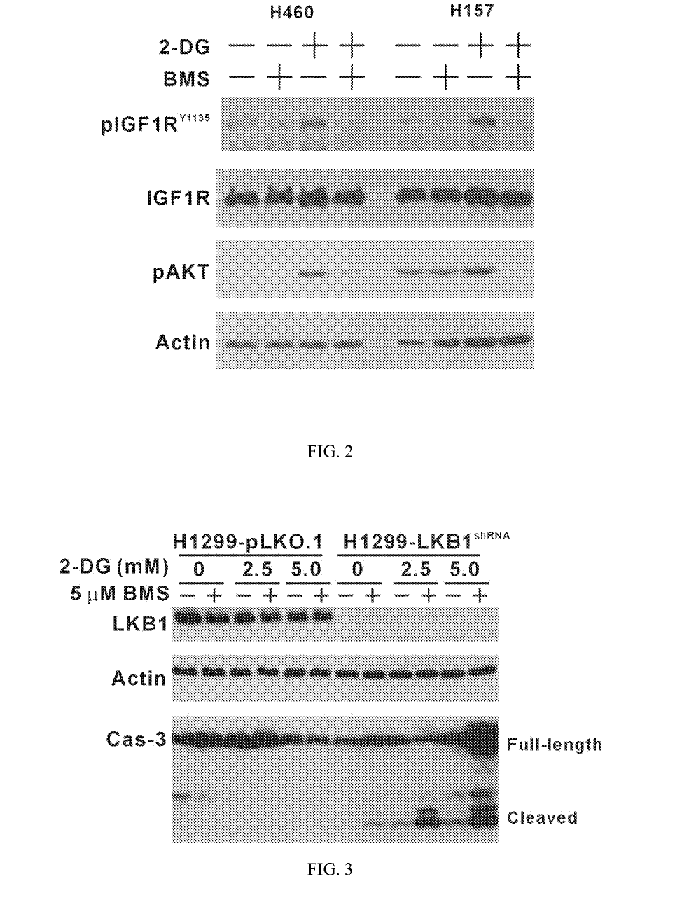

It has been discovered that BMS-754807 and 2-DG synergistically act in the suppressing of H460 and H157 cell proliferation. The inhibition of IGF1R function by an IGF1R inhibitor facilitates 2-DG medicated cell killing in LKB1-deficient cells, such as H460 and H157. The IGF1R inhibitor BMS-754807 block Akt phosphorylation induced by 2-DG (FIG. 2). Combination index analysis indicates that BMS-754807 and 2-DG had a synergistic effect on cell proliferation (Table 1).

TABLE 1
Summary of combination indexes (Cls) generated from the isobologram
at increasing concentrations of 2-DG and BMS-754807

| 2-DG | BMS-754807 | | | | |
|---|---|---|---|---|---|
| (mM) | FA_{2-DG} | (uM) | FA_{BMS-754807} | FA_{comb} | Cl |
| | | | | H460 | |
| 2.5 | 0.95 | 5.0 | 0.80 | 0.63 | 0.220 |
| 2.5 | 0.95 | 10.0 | 0.69 | 0.43 | 0.130 |
| 5.0 | 0.81 | 5.0 | 0.80 | 0.49 | 0.300 |
| 5.0 | 0.81 | 10.0 | 0.69 | 0.38 | 0.220 |
| 10.0 | 0.71 | 5.0 | 0.80 | 0.40 | 0.460 |
| 10.0 | 0.71 | 10.0 | 0.69 | 0.25 | 0.290 |
| | | | | H157 | |
| 2.5 | 0.930 | 5.0 | 0.990 | 0.740 | 0.330 |
| 2.5 | 0.930 | 10.0 | 0.820 | 0.670 | 0.260 |
| 5.0 | 0.800 | 5.0 | 0.990 | 0.660 | 0.490 |
| 5.0 | 0.800 | 10.0 | 0.820 | 0.580 | 0.380 |
| 10.0 | 0.690 | 5.0 | 0.990 | 0.570 | 0.720 |
| 10.0 | 0.690 | 10.0 | 0.820 | 0.440 | 0.480 |

Cl less than 1.0 indicates synergy, while Cl greater than 1.0 indicates antagonism.
FA_{2-DG} indicates fraction growth inhibition by 2-DG alone; FA_{BMS-745807}, fraction growth inhibition by BMS-745807 alone; and FA_{Comb}, fraction growth inhibition by 2-DG and BMS-754807.

### Method of Managing Cancer

It is contemplated that any type of tumor and any type of tumor antigen may be targeted with the corresponding biology of the therapeutics disclosed herein. The cancer can be one or more of, for example, breast cancer, colon cancer, ovarian carcinoma, osteosarcoma, cervical cancer, prostate cancer, lung cancer, synovial carcinoma, pancreatic cancer, melanoma, multiple myeloma, neuroblastoma, and rhabdomyosarcoma, or other cancer yet to be determined in which IGF-IR levels are elevated, up-regulated, mutated or altered in physiology compared to non-oncogenic cells.

Other exemplary types of tumors that may be targeted include acute lymphoblastic leukemia, acute myelogenous leukemia, biliary cancer, breast cancer, cervical cancer, chronic lymphocytic leukemia, chronic myelogenous leukemia, colorectal cancer, endometrial cancer, esophageal, gastric, head and neck cancer, Hodgkin's lymphoma, lung cancer, medullary thyroid cancer, non-Hodgkin's lymphoma, multiple myeloma, renal cancer, ovarian cancer, pancreatic cancer, glioma, melanoma, liver cancer, prostate cancer, and urinary bladder cancer.

Additionally, tumor-associated targets may be targeted. In some embodiments antigen targeting will help localize the therapeutic in terms of tissue distribution or increased local concentration affect either in the tissue or desired cell type. Alternatively, it may provide an additional mechanism of action to combat cancer along with one of the targets described herein for which a therapeutic is made. Such antigens or targets include, but are not limited to, carbonic anhydrase IX, A3, antigen specific for A33 antibody, BrE3-antigen, CD1, CD1a, CD3, CD5, CD15, CD16, CD19, CD20, CD21, CD22, CD23, CD25, CD30, CD45, CD74, CD79a, CD80, HLA-DR, NCA 95, NCA90, HCG and its subunits, CEA (CEACAM-5), CEACAM-6, CSAp, EGFR, EGP-1, EGP-2, Ep-CAM, Ba 733, HER2/neu, hypoxia inducible factor (HIF), KC4-antigen, KS-1-antigen, KS1-4, Le-Y, macrophage inhibition factor (MIF), MAGE, MUC1, MUC2, MUC3, MUC4, PAM-4-antigen, PSA, PSMA, RS5, S100, TAG-72, p53, tenascin, IL-6, IL-8, insulin growth factor-I (IGF-I), insulin growth factor-II (IGF-II), Tn antigen, Thomson-Friedenreich antigens, tumor necrosis antigens, placenta growth factor (P1GF), 17-IA-antigen, an angiogenesis marker (e.g., ED-B fibronectin), an oncogene marker, an oncogene product, and other tumor-associated antigens. Recent reports on tumor associated antigens include Mizukami et al., (2005, Nature Med. 11:992-97); Hatfield et al., (2005, Curr. Cancer Drug Targets 5:229-48); Vallbohmer et al. (2005, J. Clin. Oncol. 23:3536-44); and Ren et al. (2005, Ann. Surg. 242:55-63), each incorporated herein by reference.

### Formulation and Administration

Therapeutic formulations are prepared for storage by mixing the described proteins having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants.

The formulations herein may also contain more than one active compound as necessary for the particular indication being treated, preferably those with complementary activities that do not adversely affect each other. Examples of combinations of active compounds are provided in herein. Such molecules are suitably present in combination in amounts that are effective for the purpose intended.

Another embodiment of the disclosure provides methods for the treatment of a subject having a cancer by administering a IGF-IR inhibitor and 2-deoxyglucose, either alone or in combination with other cytotoxic or therapeutic agents. In particular, preferred cytotoxic and therapeutic agents include docetaxel, paclitaxel, doxorubicin, epirubicin, cyclophosphamide, trastuzumab, capecitabine, tamoxifen, toremifene, letrozole, anastrozole, fulvestrant, exemestane, goserelin, oxaliplatin, carboplatin, cisplatin, dexamethasone, antide, bevacizumab, 5-fluorouracil, leucovorin, levamisole, irinotecan, etoposide, topotecan, gemcitabine, vinorelbine, estramustine, mitoxantrone, abarelix, zoledronate, streptozocin, rituximab, idarubicin, busulfan, chlorambucil, fludarabine, imatinib, cytarabine, ibritumomab, tositumomab, interferon alpha-2b, melphalam, bortezomib, altretamine, asparaginase, gefitinib, erlonitib, anti-EGF receptor antibody (e.g., cetuximab or panitumumab), ixabepilone, and an epothilone or derivative thereof. More preferably, the therapeutic agent is a platinum agent (such as carboplatin, oxaliplatin, cisplatin), a taxane (such as paclitaxel, docetaxel), gemcitabine, or camptothecin.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the proteins of the disclosure, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers, (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated proteins of the disclosure may remain in the body for a long time, they may denature or aggregate as a result of exposure to moisture at 37 C, resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

For therapeutic applications, the compounds disclosed herein may administered to a subject, in a pharmaceutically acceptable dosage form. They can be administered intravenously as a bolus or by continuous infusion over a period of time, by intramuscular, subcutaneous, intra-articular, intrasynovial, intrathecal, oral, topical, or inhalation routes. They may also be administered by intratumoral, peritumoral, intralesional, or perilesional routes, to exert local as well as systemic therapeutic effects. Suitable pharmaceutically acceptable carriers, diluents, and excipients are well known and can be determined by those of skill in the art as the clinical situation warrants. Examples of suitable carriers, diluents and/or excipients include: (1) Dulbecco's phosphate buffered saline, pH about 7.4, containing about 1 mg/ml to 25 mg/ml human serum albumin, (2) 0.9% saline (0.9% w/v NaCl), and (3) 5% (w/v) dextrose. Methods can be practiced in vitro, in vivo, or ex vivo.

### Terms

As used herein, “alkyl” means a noncyclic straight chain or branched, unsaturated or saturated hydrocarbon such as those containing from 1 to 10 carbon atoms, while the term “lower alkyl” or “C₁₋₄alkyl” has the same meaning as alkyl but contains from 1 to 4 carbon atoms. The term “higher alkyl” has the same meaning as alkyl but contains from 7 to 20 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, “heteroaryl” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above attached through a sulfur bridge. An example of an alkylthio is methylthio, (i.e., —S—CH₃).

“Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above attached through an amino bridge. An example of an alkylamino is methylamino, (i.e., —NH—CH₃).

“Alkanoyl” refers to an alkyl as defined above attached through a carbonyl bride (i.e., —(C═O)alkyl).

“Alkylsulfonyl” refers to an alkyl as defined above attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfinyl” refers to an alkyl as defined above attached through a sulfinyl bridge (i.e. —S(═O)alkyl).

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In preferred embodiment the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Subject” refers any animal, preferably a human patient, livestock, rodent, monkey or domestic pet.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur or nitrogen atom or replacing an amino group with a hydroxyl group or vice versa. The derivative may be a prodrug. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, her2 for breast cancer, or others.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the time to disease progression (TTP) and/or determining the response rates (RR).

### Experimental

### EXAMPLE 1

### IC₅₀ of BMS754807 and 2-DG in LKB1-Null NSCLC Cell Lines

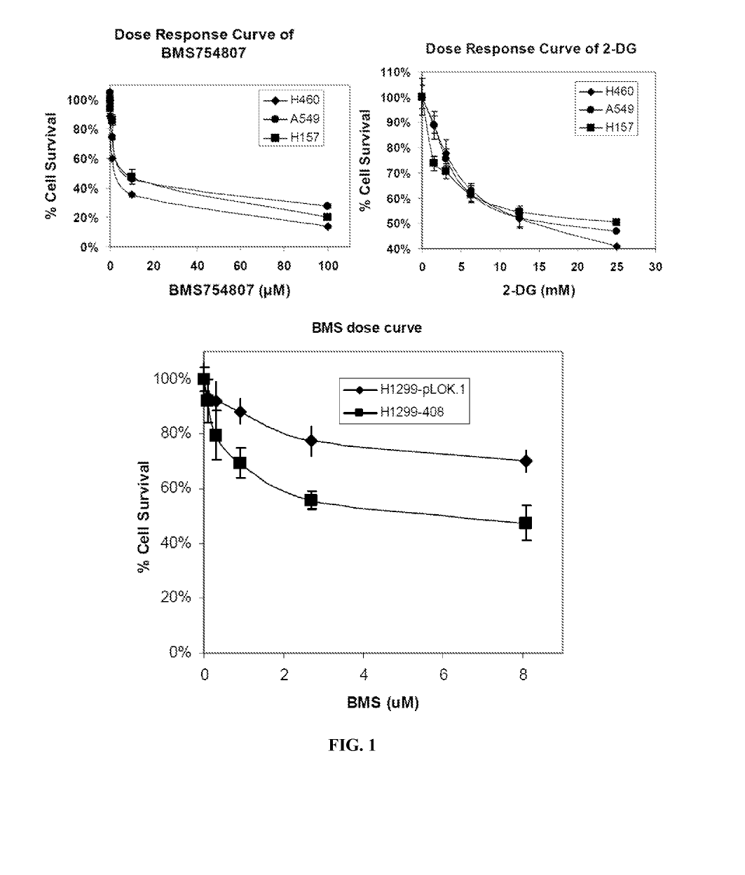

Analysis focused on NSCLC cell lines with LKB1 inactivation mutation. A549 and H460 both contain a Q37 to nonsense mutation, and H157 contains exon 2 and 3 deletion. All these mutations results in the loss of LKB1 protein function. MTS assay was used to measure the IC50 of BMS754807 and 2-DG in these cells (FIG. 1). IC₅₀ of BMS754807 for A549, H460, and H157 are 16.6, 3.25 and 10.8 μM, respectively, and IC₅₀ of 2-DG for A549, H460, and H157 are 15.7, 14.1 and 21.8 mM, respectively. See FIG. 1.

### EXAMPLE 2

### BMS754807 Blocked 2-DG Induced IGF1R and Akt Phosphorylation

Whether 2-DG induced AKT phosphorylation can be blocked by BMS754807 was determined. LKB1-null NSCLC cell lines was pre-incubated with 10 μM of BMS754807 for 30 minutes, and then stimulated with 25 mM 2-DG to induce phosphorylation. BMS754807 blocked 2-DG induced IGF1R and AKT phosphorylation in H460, and H157 cells. Therefore, BMS754807 effectively inhibited IGF1R-mediated AKT phosphorylation in both cell lines. See FIG. 2.

### EXAMPLE 3

### The Combination of 2-DG and BMS754807 to Initiates Caspase-3 Cleavage in LKB1-Depleted Cells

Whether apoptosis was induced by the combination of 2-DG and BMS754807 was evaluated. Here, LKB1 was depleted in H1299 cells by shRNA. Cells were treated with 2-DG and BMS754807for 72 hours, and caspase-3 cleavage was used as a surrogate marker for the induction of apoptosis. The combination of 2-DG and BMS754807 induced caspase-3 cleavage specifically in LKB1-depleted H1299 cells. See FIG. 3.

1. A method of treating cancer comprising administering to a subject a pharmaceutical compositions comprising 2-deoxyglucose and administering a pharmaceutical composition comprising an IGF-IR inhibitor.

2. The method of claim 1, wherein the IGF-IR inhibitor is 1-(4-((5-cyclopropyl-1H-pyrazol-3-yl)amino)pyrrolo[2,1-f][1,2,4]triazin-2-yl)-N-(6-fluoropyridin-3-yl)-2-methylpyrrolidine-2-carboxamide or salt thereof.

3. The method of claim 1, wherein the 2-deoxyglucose and the IGF-IR inhibitor are in the same pharmaceutical composition.

4. The method of claim 1, wherein said subject is diagnosed with breast cancer, colon cancer, prostate cancer, ovarian carcinoma, synovial sarcoma, pancreatic cancer, lung cancer, osteosarcoma, or cervical cancer.

5. The method of claim 1, wherein another anticancer agent is administered to the subject.

6. A pharmaceutical composition comprising 2-deoxyglucose and an IGF inhibitor and a pharmaceutically acceptable excipient.

7. The pharmaceutical composition of claim 5, wherein the IGF inhibitor is 1-(4-((5-cyclopropyl-1H-pyrazol-3-yl)amino)pyrrolo[2,1-f][1,2,4]triazin-2-yl)-N-(6-fluoropyridin-3-yl)-2-methylpyrrolidine-2-carboxamide or salt thereof.
